(19) United States
(12) Patent Application Publication
(10) Pub. No.: US 2017/0224515 A1
(43) Pub. Date: Aug. 10, 2017

(54) JOINT MOVEMENT SUPPORTING PROTECTION DEVICE

(71) Applicant: Hongtao YUAN, Changsha City (CN)

(72) Inventor: Hongtao YUAN, Changsha City (CN)

(21) Appl. No.: 15/328,654

(22) PCT Filed: Jul. 28, 2015
(86) PCT No.: PCT/CN2015/085291

§ 371 Date: Jan. 24, 2017

(30) Foreign Application Priority Data
Jul. 29, 2014 (CN) 201410365832.3

(51) Int. Cl. A61F 5/01; A63B 71/08

(52) U.S. Cl. CPC A61F 5/013; A61F 5/01; A61F 5/0123; A63B 71/08; A61F 2005/0165

(57) ABSTRACT

A joint movement supporting protection device, comprising a joint protection body (1) matched with a joint in shape, and a fixing device (2) connected or contacted with the joint protection body (1) and tightly attaching the joint protection body (1) to a joint of a human body. A plurality of upper notches (11) extending downwards from top but not penetrating through bottom are formed in a portion, close to a joint movement area, of the joint protection body (1). Thus, the joint protection body (1) is divided into a plurality of joint pieces (12). A junction of the upper notch (11) and a lower edge of the joint protection body (1) serves as a rotation fulcrum (13) between adjacent joint pieces (12). When the joint bends to a limiting position, the two adjacent joint pieces (12) on two sides of the upper notch (11) contact.

### FIELD OF THE INVENTION

The present disclosure relates to the field of human body movement protection device, and particular to a joint movement supporting protection device.

### BACKGROUND OF THE INVENTION

At present, joint protection devices are mainly divided into two types: elastic fiber fabric protection devices with rubber bands and plastic supporting protection devices. Common elastic fiber fabric protection devices with rubber bands include ankle protection devices, wrist protection devices, knee protection devices, waist protection devices, finger protection devices and so on. Their principles are that material elasticity is utilized to increase joint stability so as to buffer and prevent exterior injury and excessive deformation to some extent. However, since the elastic fiber fabric protection devices with rubber bands are simplex in material and structure, so the physical performance in all directions are identical, and thus they are limited in protection of joints under the requirement of giving consideration to joint movement functions. The reason is very simple: binding strength needs to be enhanced to increase protection to joints, which may have a negative effect on joint movement; instead, protection to the joints may be reduced if the binding strength is reduced to take the joint movement into account. Common plastic supporting protection devices include knee protection devices in skating and skiing sports, and finger joint supporting protection devices in football goalkeeper gloves. This type of protection devices make up for some deficiencies of the former type of protection devices. However, at present, this type of protection devices are larger in structural volume, unsuited to bodies, uncomfortable for wearing, complex in structure, and thus relatively high in cost.

To a basketball fan, finger injury is not fatal as knee injury or ankle injury. However, fingers are slender and vulnerable, unable to bear great strength, and are in contact with a basketball momently. Therefore, fingers are more susceptible to get injured. Once the fingers are injured, a seriously negative effect may be undoubtedly caused on the basketball sport. Injured fingers may be more vulnerable, and thus may be easily susceptible to repeated injury, which causes the injured fingers hardly be restored for a long time. In the field of basketball sports, a basketball finger protection device that can provide effective protection to fingers has been always expected to appear. However, at present, there is only one type of finger protection device in the market, namely, an elastic barrel-shaped finger protection device with a rubber band. However, this type of finger protection device is not comfortable and is limited in protection of fingers. Therefore, it is still a problem to be solved in the art to develop a finger protection device that can provide effective protection to fingers and is comfortable.

### SUMMARY OF THE INVENTION

A technical problem solved by the present disclosure resides in overcoming the above defects and providing a joint movement supporting protection device which is light and thin in volume, simple in structure, can be firmly attached to a joint part, does not affect normal joint movement, and is low in cost.

Embodiments of the invention provide a joint movement supporting protection device, including a joint protection body matched with a joint in shape, and a fixing device in connection or contact with the joint protection body and tightly attaching the joint protection body to a joint of a human body, wherein a plurality of upper notches extending downwards from top are formed in a portion, close to a joint movement area, of the joint protection body, a plurality of joint pieces are formed between a plurality of the upper notches; a junction between the upper notch and a lower edge of the joint protection body constitutes a rotation fulcrum between two adjacent joint pieces; when the joint bends to a limiting position, the two adjacent joint pieces on two sides of the upper notch contact. Further, lower notches are further formed in positions, extending downwards from a bottom side of the upper notches, the lower notches correspond to the upper notches one by one, a depth of the lower notch is less than a depth of the upper notch, a junction between the upper notch and the lower notch constitutes a rotation fulcrum between two adjacent joint pieces.

Further, a ratio of the depth of the lower notch to the depth of the upper notch is 1:2˜1:8.

Further, edges of the upper notch or the lower notch and the joint piece are rounded off.

Preferably, bottom width of the upper notch is larger than top width thereof.

Preferably, heights of a plurality of the joint pieces increase progressively, and each joint piece extends outwardly and covers over top of the upper notch.

Preferably, the fixing device includes a plurality of strip-shaped fixing belts, an end of the fixing belt and a side of the joint protection body are integrally molded, a tail end of the fixing belt is provided with at least one buckle, the joint protection body is provided with a through hole matched with the buckle; after wrapping the joint, the fixing belt is fastened to the joint protection body by fastening the buckle to the through hole.

Further, a tail end of the fixing belt may be further provided with at least one saw-toothed barb, and the joint protection body is provided with a corresponding through hole or semi-through hole matched with the saw-toothed barb.

Preferably, the fixing device includes fixing rings arranged on two ends of the joint protection body; the fixing rings are integrally molded with the joint protection body.

Further, the fixing device is a closed ring or a split ring.

Based on the above technical features, the joint movement supporting protection device proposed in the embodiments of the invention can be firmly attached to a joint part, does not affect the normal joint movement, and effectively protect joints; moreover, it is simple in structure and low in cost, and can be efficiently and conveniently worn and detached.

### BRIEF DESCRIPTION OF THE DRAWINGS

FIG. 1 is a front perspective view of a joint movement supporting protection device according to the present disclosure;

FIG. 2 is a side perspective view of the joint movement supporting protection device according to the present disclosure;

FIG. 3 is a side sectional view of the joint movement supporting protection device according to the present disclosure;

FIG. 4 is a use state diagram I of the joint movement supporting protection device according to the present disclosure;

FIG. 5 is a use state diagram II of the joint movement supporting protection device according to the present disclosure;

FIG. 6 is a sectional view of a fastened fixing belt of the joint movement supporting protection device according to the present disclosure;

FIG. 7 is a side view of the joint movement supporting protection device according to the present disclosure; and

FIG. 8 is a partial sectional view of the joint movement supporting protection device according to the present disclosure; and

FIG. 9 is a side perspective view of a joint movement supporting protection device according to another embodiment of the present disclosure; and

FIG. 10 is a side view of a joint movement supporting protection device according to another embodiment of the present disclosure; and

FIG. 11 is a front perspective view of a joint movement supporting protection device according to another embodiment of the present disclosure; and

FIG. 12 is a side perspective view of a joint movement supporting protection device according to another embodiment of the present disclosure; and

FIG. 13 is a front perspective view of a joint movement supporting protection device according to another embodiment of the present disclosure; and

FIG. 14 is a front perspective view of a joint movement supporting protection device according to another embodiment of the present disclosure; and

FIG. 15 is a use diagram I of a joint movement supporting protection device according to another embodiment of the present disclosure;

FIG. 16 is a use diagram II of a joint movement supporting protection device according to another embodiment of the present disclosure.

### DETAILED DESCRIPTION OF THE EMBODIMENTS

In order to make the aims, technical solutions and advantages of the present invention clear, the technical solution of the present disclosure is described in detail in the following with reference to specific embodiments and drawings of specification. It is understood that the following specific embodiments are merely illustrative and not for limiting.

The implementation of the present disclosure is described in detail in the following with reference to specific embodiments.

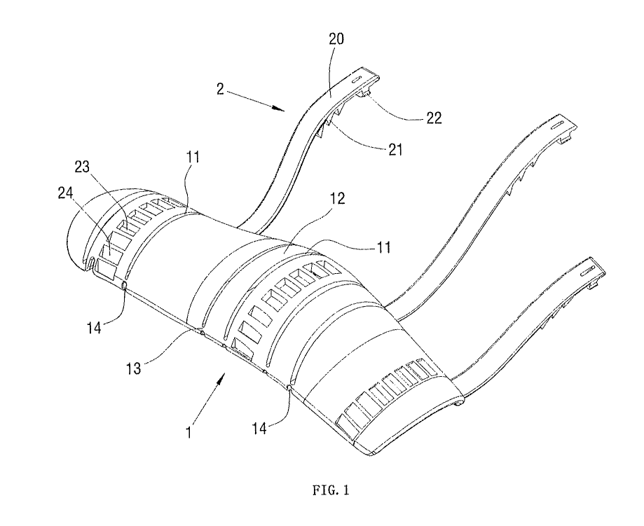

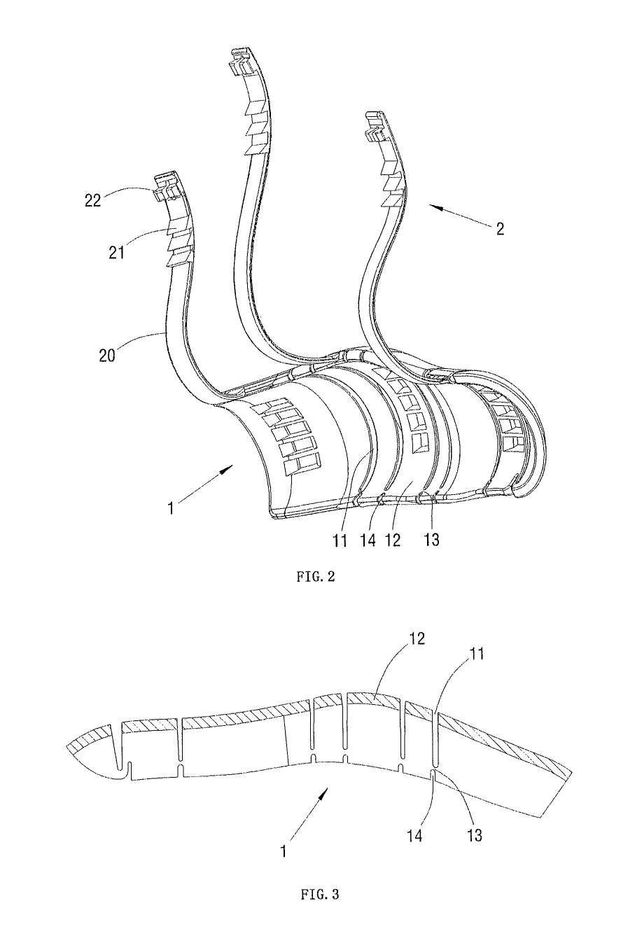

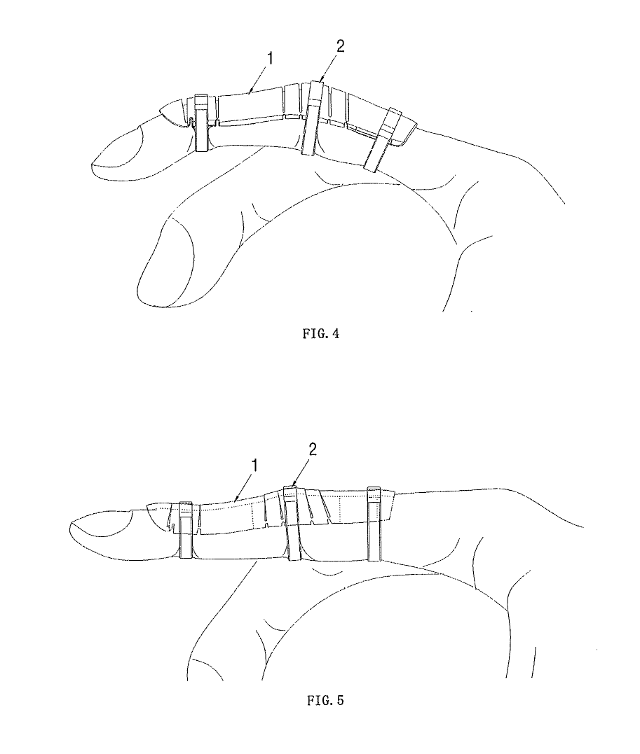

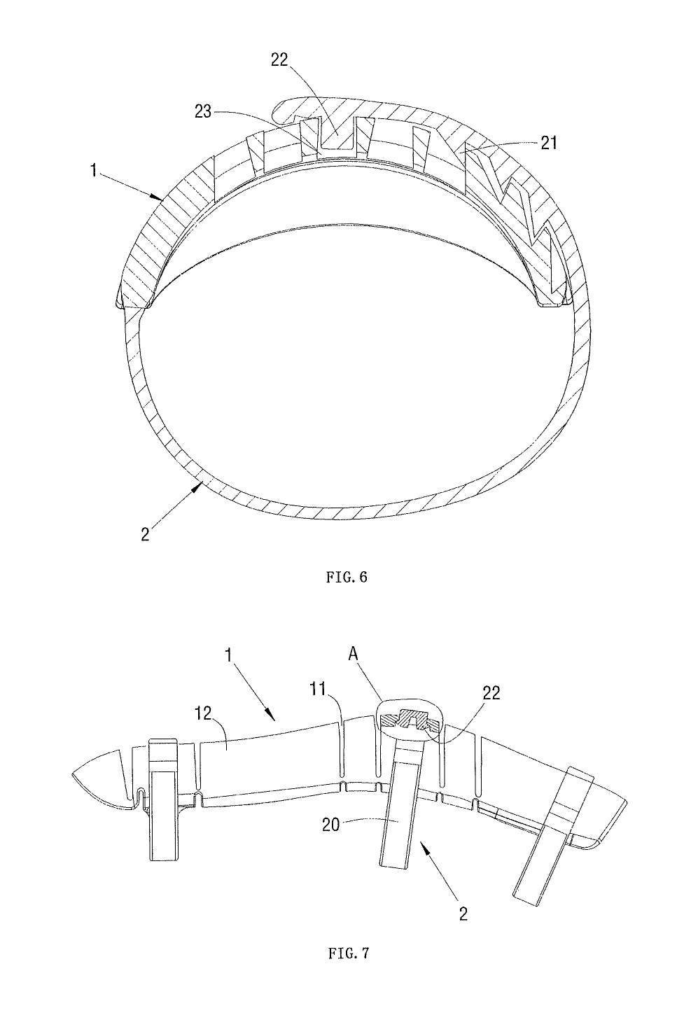

As shown in FIG. 1 to FIG. 8, embodiments of the present disclosure provide a joint movement supporting protection device, including a joint protection body 1, whose cross section is arc-like, matched with a joint in shape, size and length, and a fixing device 2 in connection or contact with the joint protection body and tightly attaching the joint protection body to a joint of a human body. Specifically, a plurality of upper notches 11 extending downwards from top but not penetrating through bottom are formed in a portion, close to a joint movement area, of the joint protection body 11, thus the joint protection body 1 is divided, by the upper notches 11, into a plurality of joint pieces 12, i.e. a plurality of joint pieces 12 are formed among a plurality of upper notches 11; at the same time, a junction between the upper notch 11 and a lower edge of the joint protection body 1 constitutes a rotation fulcrum 13 between adjacent two joint pieces 12; the adjacent joint pieces 12 can rotate around the rotation fulcrum 13, and when people wear the joint movement supporting protection device and keep the fingers straight, the adjacent joint pieces 12 do not rotate any more after they come into contact with each other. In this way, the joint is prevented from overbending toward a direction to back of a hand, thereby achieving the aim of protecting a finger. Regarding to finger joints, most importantly, the finger needs to be prevented from overbending toward the back of the finger, which is a primary cause to injury to the finger joints. The case of the above embodiment may be adopted when restricting the finger joints from bending toward a direction of a palm of the hand is left out of account.

In an embodiment of the present disclosure, lower notches 14 are further formed in positions, extending downwards from a bottom side, of corresponding positions of the upper notches 11, the lower notches 14 correspond to the upper notches 11, a depth of the lower notch 14 is less than a depth of the upper notch 11, specifically, an optional range is as below: a ratio of the depth of the lower notch 14 to the depth of the upper notch 11 is 1:2˜1:8, thus the joint protection body 1 is divided, by the upper notches 11 and the lower notches 14, into a plurality of joint pieces 12, and a junction between the upper notch 11 and the lower notch 14 constitutes a rotation fulcrum 13 between adjacent joint pieces. Two adjacent joint pieces of the upper notch 11 just come into contact with each other when the joint is straightened, at the moment the adjacent joint pieces 12 do not rotate anymore because of a rigid support action of the joint protection body, so that the joint is prevented from overbending toward the direction of the back of the hand, thereby achieving the aim of protecting the finger. When the joint bends toward the direction of the palm of the hand to a certain extent, two adjacent joint pieces 12 of the lower notch 14 just come into contact with each other, at the moment the adjacent joint pieces 12 of the lower notch 14 do not rotate anymore because of a rigid support action of the joint protection body, so that the joint is prevented from overbending toward the direction of the palm of the hand, thereby achieving the aim of protecting the finger.

In an embodiment of the present disclosure, edges of the upper notch 11 or the lower notch 14 and the joint piece 12 are rounded off. Here, edges of the upper notch 11 or the lower notch 14 and the joint piece 12 are rounded off to improve fitness to the human body and performance.

In another embodiment of the present disclosure, as shown in FIG. 10, bottom width of the upper notch 11 is larger than top width thereof, so that rotating part connecting the adjacent two joint pieces 12 is stronger and not easy to break, thus the overall resilience of the joint movement supporting protection device is improved. Of course, according to actual demands, in other embodiment, it is possible to take some other means to improve the overall resilience of the joint movement supporting protection device.

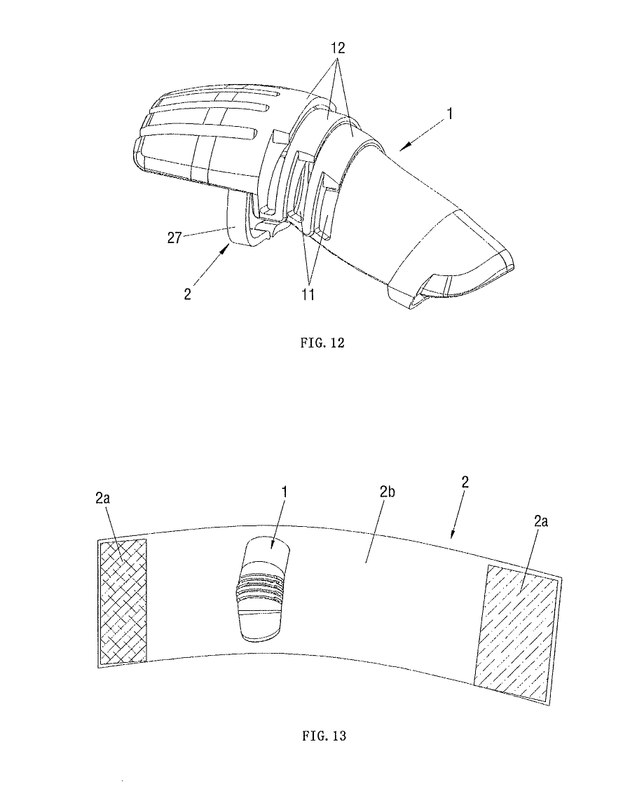

In an embodiment of the present disclosure, as shown in FIG. 12, heights of a plurality of the joint pieces 12 increase progressively, and each joint piece 12 extends outwardly and covers over top of the upper notch 11, thus, when finger joints are bend to some extent in a direction towards palm, the adjacent two joint pieces 12 rotate reversely around the rotation fulcrum 13, then, top of the upper notch 11 opens, since edge of each joint piece 12 extends outwards and cover over top of the upper notch 11, top of the upper notch 11 will not expose entirely, preventing injury from external objects to fingers or invasion of external objects, thus the overall safety of the joint movement supporting protection device is improved.

In an embodiment of the present disclosure, a plurality of strip-shaped fixing belts 20 are adopted for the fixing device 2, an end of the fixing belt 20 and a side of the joint protection body 1 are integrally molded, a tail end of the fixing belt 20 is provided with at least one buckle 22, correspondingly, the joint protection body 1 is provided with a plurality of through holes 23 matched with the buckle 22; after wrapping the joint, the fixing belt 2 is fastened to the joint protection body 1 by fastening the buckle 22 to the through holes 23 disposed on the joint protection body 1 so that the joint movement supporting protection device is tightly fastened to the joint. Of course, according to actual demands, in other embodiment, the fixing device 2 can have some other structure, and it is not limited.

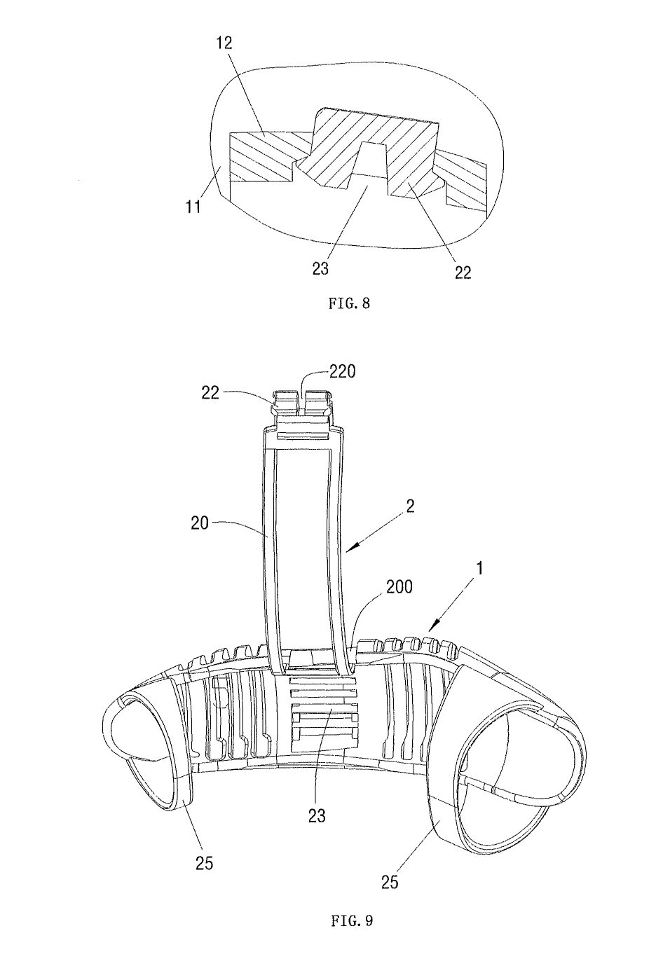

In an embodiment of the present disclosure, an end of the fixing belt 2 may be further provided with at least one saw-toothed barb 21, correspondingly, the joint protection body 1 is provided with a plurality of through holes 23 or semi-through holes 24 matched with the saw-toothed barb 21, so that the saw-toothed barb 21 is fastened to the through hole 23 or semi-through hole 24. FIG. 7 and FIG. 8 display a detail state that the buckle 22 is fastened to the through hole 23. The saw-toothed barb 21 and the buckle 22 cooperate with different through holes 23 or semi-through holes 24 so that a degree of tightness of the fixing belt 2 can be adjusted. In this way, the same joint protection body can fit to fingers of different sizes to a certain extent.

In other embodiments of the present disclosure, according to requirements for fixation of protection devices in different fields, a suitable structural style adopting other types may be selected for the fixing device 2. For example, a traditional Velcro tape is adopted for fixation, the joint protection body 1 may be inlaid and fixed into a glove or an elastic fabric, or can be fixed by an independent fixing belt, which can adopt a material in a proper form and match with a buckle having a proper structure to fasten the joint protection body 1. In this way, the joint protection body 1 is fastened to the joint of the human body.

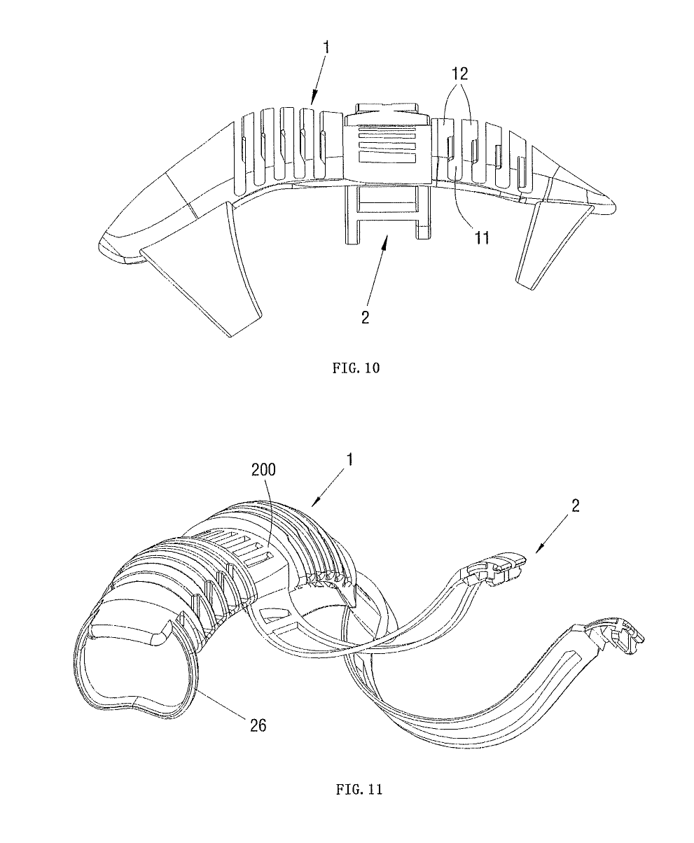

As shown in FIG. 9, in another embodiment of the present disclosure, the above fixing device 2 may include the above fixing belt 20 and a fixing ring 25 arranged on two ends of the joint protection body 1. The fixing ring 25 is integrally molded with the joint protection body 1 to form a soft plastic annular structure, so that the fixing ring 25 and the joint protection body 1 form one piece. At the same time, the joint protection body 1 is provided with a containing slot 200 to accommodate the fixing belt 20 on the back, i.e. when the fixing belt 20 surrounds the back of the joint protection body 1, the outer end portion of the fixing belt 20 is accommodated in the containing slot 200, so that the height of the fixing belt 20 is flush with the back of the joint protection body 1, avoiding being unaesthetic and scratch; furthermore, an opening 220 is provided at the middle of the buckle 22, thanks to the opening 220, the buckle 22 can access to the above through hole 23 more easily, improving convenience to take on and off.

Alternatively, as shown in FIG. 11, the fixing device 2 further includes a closed annular fixing structure formed from a single material by single shot shaping technique, i.e. a closed ring 26 is formed between the fixing device 2 and the joint protection body 1, and as the material of the above joint protection body 1, the fixing device 2 is formed by single shot shaping technique. Here, the closed ring 26 is set to be corrugation, so that it can adapt to joint of different sizes, broadening the application of the joint movement supporting protection device and meeting people's different demands. At the same time, the joint protection body 1 is also provided with a containing slot 200 to accommodate the fixing belt 20 on the back, avoiding being unaesthetic and scratch.

Alternatively, as shown in FIG. 12, the fixing device 2 further includes a split annular fixing structure formed from a single material by single shot shaping technique, i.e. a split ring 27 is formed between the fixing device 2 and the joint protection body 1, and as the material of the above joint protection body 1, the fixing device 2 is formed by single shot shaping technique. Thus, it can adapt to joint of different sizes, broadening the application of the joint movement supporting protection device and meeting people's different demands.

Alternatively, the joint protection body 1 can be fixed on joint by the following means: the joint protection body 1 matched with the shape and structure of joint to be protected may be inlaid or fixed into a fabric, and then the fabric is wrapped around the joint and fixed by Velcro. Specifically, as shown in FIG. 13, for example, in order to protect knee-joint, the fixing device 2 includes Velcro 2a and fabric 2b, and the joint protection body 1 is disposed on fabric 2b, when in use, the joint protection body 1 is positioned on the knee-joint, then the fabric 2b is wrapped around the leg, and at last, Velcro 2a on two ends of the fabric 2b are engaged.

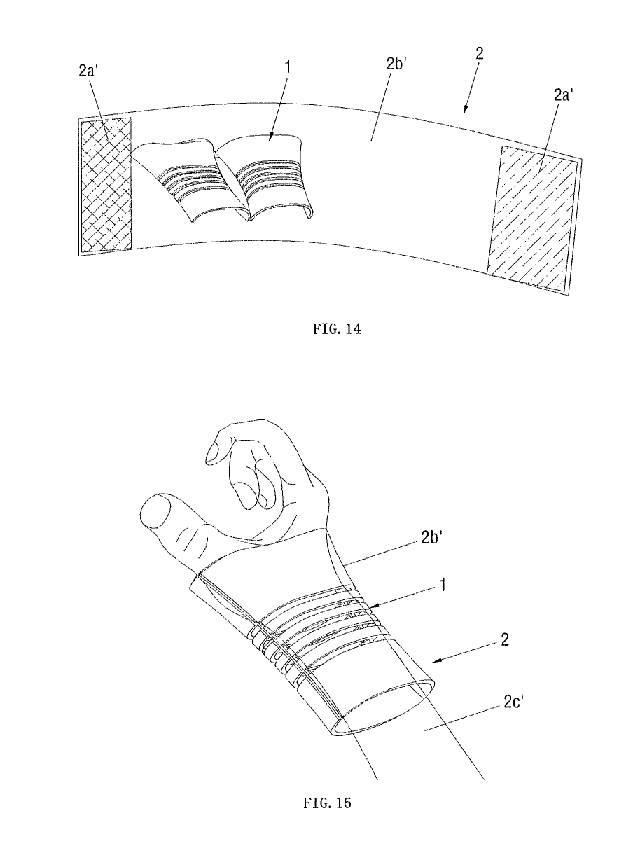

Furthermore, if people need to restrict movement of joint in multiple directions, a plurality of joint protection bodies 1 can be used, i.e. the joint protection body 1 matched with the shape and structure of joint to be protected may be inlaid or fixed into a fabric with certain shape and size, and then the fabric is wrapped around the joint and fixed by Velcro. Specifically, as shown in FIGS. 14-15, for example, in order to protect wrist joint, the fixing device 2 includes Velcro 2a′ and fabric 2b′, and two joint protection bodies 1 is disposed on fabric 2b′, when in use, the two joint protection bodies 1 are positioned on the wrist joint of an arm 2c′ oppositely, then the fabric 2b′ is wrapped around the arm 2c′, and at last, Velcro 2a′ on two ends of the fabric 2b′ are engaged. The protection device can applied to some other body parts, such as elbow, ankle, waist, wherein the structures and principles are the same, but different shapes should be designed for different body parts to be protected.

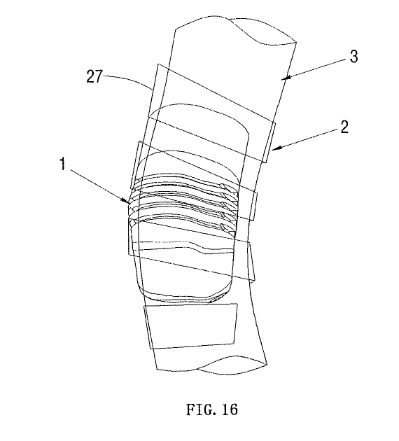

Alternatively, the above joint protection body 1 can be used in combination with self-adhesive non-woven fabrics or various tape or gummed paper or some other known fixing means, directly applied to protecting devices of exercise or medical rehabilitation devices. For example, in order to protect knee-joint, see FIG. 16, the fixing device 2 includes self-adhesive non-woven fabric 27 (or tape or gummed paper), and the joint protection body 1 is positioned tightly on knee-joint 3, then joint protection body 1 is fixed on the knee-joint 3 through self-adhesive non-woven fabric.

In other embodiments of the present disclosure, the above joint protection body 1 can also be inlaid into shoes in a position where some parts need to be protected, preventing ankle from being sprained without obstructing movement of feet; alternatively, the above joint protection body 1 can also be inlaid into sporting gloves (such as goalkeeper's gloves) as a reinforce, to withstand the impact when hands catch a football, to prevent injuries to the fingers; alternatively, the above joint protection body 1 can also be applied as an internal layer of surface decorative parts of artificial limbs, as a support of the decorative parts; through the joint protection body 1, the structures of the artificial limbs can be simplified, making it more aesthetic, while keeping it flexible.

Application of the patent structure to finger protection in basketball is one of embodiments most fully reflecting advantages of the patent structure. Therefore, this patent is expounded mainly by means of an embodiment in which the joint movement supporting protection device is applied to finger protection in basketball. To applications in other aspects such as a football goalkeeper glove support, finger protection for playing musical instruments, finger protection for vegetable cutting, and even protection of wrists, ankles and knees or the like in ball games and outdoor sports, their structures and principles are roughly identical, merely shapes, sizes and so on are different. Therefore, these applications all fall within the scope of protection of this patent.

Based on the above technical features, the joint movement supporting protection device proposed in the embodiments of the invention can be firmly attached to a joint part, does not affect the normal joint movement, and effectively protect joints; moreover, it is simple in structure and low in cost, and can be efficiently and conveniently worn and detached.

Preferable embodiments of the invention are specifically described above, but the invention is not limited to the embodiments, the skilled familiar to the art can make various equivalent transformations and substitutions without departing from the spirit of the invention and these transformations and substitutions fall within the scope defined by claims.

1: A joint movement supporting protection device, comprising a joint protection body matched with a joint in shape, and a fixing device in connection or contact with the joint protection body and tightly attaching the joint protection body to a joint of a human body, characterized in that: a plurality of upper notches are formed in a portion, close to a joint movement area, of the joint protection body; the upper notches divide the joint protection body into a plurality of joint pieces with some connecting parts connecting adjacent joint pieces together, and adjacent joint pieces can rotate around the connecting parts as fulcrums; when the joint pieces rotate to a limiting position around the fulcrums, the joint pieces do not rotate any more since adjacent joint pieces contact to each other.

2: The joint movement supporting protection device according to claim 1, characterized in that corresponding lower notches are further formed in positions, extending downwards from a bottom side, of corresponding positions of the upper notches, the lower notches correspond to the upper notches one by one, a depth of the lower notch is less than a depth of the upper notch, and a junction between the upper notch and the lower notch constitutes a rotation fulcrum between adjacent joint pieces.

3: The joint movement supporting protection device according to claim 2, characterized in that a ratio of the depth of the lower notch to the depth of the upper notch is 1:2˜1:8.

4: The joint movement supporting protection device according to claim 2, characterized in that edges of the upper notch or the lower notch and the joint piece are rounded off.

5: The joint movement supporting protection device according to claim 1, characterized in that bottom width of the upper notch is larger than top width thereof.

6: The joint movement supporting protection device according to claim 1, characterized in that heights of a plurality of the joint pieces increase progressively, and each joint piece extends outwardly and covers over top of the upper notch.

7: The joint movement supporting protection device according to claim 1, characterized in that a plurality of strip-shaped fixing belts are adapted for the fixing device, an end of the fixing belt and a side of the joint protection body are integrally molded, a tail end of the fixing belt is provided with at least one buckle, the joint protection body is provided with a corresponding through hole matched with the buckle; after wrapping the joint, the fixing belt is fastened to the joint protection body by fastening the buckle to the through hole.

8: The joint movement supporting protection device according to claim 7, characterized in that an end of the fixing belt is further provided with at least one saw-toothed barb, and the joint protection body is further provided with a corresponding through hole or semi-through hole matched with the saw-toothed barb.

9: The joint movement supporting protection device according to claim 1, characterized in that the fixing device includes fixing rings arranged on two ends of the joint protection body, the fixing rings are integrally molded with the joint protection body.

10: The joint movement supporting protection device according to claim 1, characterized in that the fixing device is a closed ring or a split ring.
